(19) United States
(12) Patent Application Publication
(10) Pub. No.: US 2021/0275024 A1
(43) Pub. Date: Sep. 9, 2021

(54) WIRELESS MEASUREMENT SYSTEM AND METHOD THEREOF FOR MEASURING DATA OF HUMAN BODY BY MULTIPLE MEASUREMENT POINTS

(71) Applicants: Inventec (Pudong) Technology Corporation, Shanghai (CN); INVENTEC CORPORATION, Taipei City (TW)

(72) Inventor: Cheng-Yu WU, TAIPEI CITY (TW)

(21) Appl. No.: 16/819,160

(22) Filed: Mar. 15, 2020

(30) Foreign Application Priority Data
Mar. 3, 2020 (CN) 202010140490.0

(51) Int. Cl. A61B 5/00; G04G 21/02; G04G 21/04; A61B 5/053; A61B 5/024; A61B 5/0408; A61B 5/0464

(52) U.S. Cl. CPC A61B 5/0022; G04G 21/025; G04G 21/04; A61B 5/0531; A61B 5/02405; A61B 5/0006; A61B 5/02427; A61B 5/0464; A61B 5/681; A61B 5/6831; A61B 5/6804; A61B 5/04085

(57) ABSTRACT

A wireless measurement system for measuring data of human body includes multiple measurement devices. One of the multiple measurement devices is a wireless measurement device configured to be attached to a skin of the human body and to obtain a first measurement data. The wireless measurement device is further configured to perform an all-day measurement and to return the first measurement data with a fixed time period when the wireless measurement device is attached to the skin of the human body.

### RELATED APPLICATIONS

This application claims priority to China Application Serial Number 202010140490.0, filed Mar. 3, 2020, which is herein incorporated by reference.

### BACKGROUND

### Field of Invention

The present disclosure relates to a wireless measurement system and a wireless measurement system for measuring data of a human bod by multiple measurement points.

### Description of Related Art

The statements in this section merely provide background information related to the present disclosure and do not necessarily constitute prior art.

Physical quantities such as voltage and impedance that changes over time in a human body may reveal many valuable information on the human body, such as health status, lifestyle, or emotional fluctuation. Some important applications can detect the early onset characteristics of heart disease. When a traditional medical equipment measures human data, such as data related to operations of heart, monitoring heart rate and heart activity is performed by measuring electrophysiological signals and electrocardiography (ECG). A method for the measurement is to connect electrodes to the human body to measure variations on electrical signals induced by heart tissues. In addition, heartbeats pass through the blood vessels with pressure, and variations of the pressure slightly change diameters of blood vessels. The variations can be measured optically by photoplethysmography (PPG). These common and important measurement techniques nowadays in hospitals are mostly performed by attaching electrodes to human skin, in which the electrodes are connected to an external receiving device via conductive wires. The data sent back to the external receiving device is collected and then interpreted by a doctor to obtain useful health information for the human body.

### SUMMARY

The above-mentioned way of measuring physical data limits physical freedom and convenience of a user to some extent, and there is really room for improvement in a modern fast-paced society. In view of this, it is an object of the present disclosure to propose a measurement system and a measurement method that can make a measurement on data of the human body more convenient and the user is free from physical constraints.

According to some embodiments of the present disclosure, a wireless measurement system for measuring data of human body is provided. The wireless measurement system includes a plurality of measurement devices. One of the plurality of the measurement devices is a wireless measurement device configured to attach a skin of the human body and to obtain a first measurement data. The wireless measurement device is further configured to perform an all-day measurement and to return the first measurement data with a fixed time period when the wireless measurement device is attached to the skin of the human body.

In one or more embodiments of the present disclosure, the plurality of the measurement devices include a wearable measurement device configured to have data communication with the wireless measurement device.

In one or more embodiments of the present disclosure, the wearable measurement device is a ribbon or a smart watch that includes at least one electrode thereon, and the at least one electrode is configured to contact the skin of the human body and to obtain a second measurement data.

In one or more embodiments of the present disclosure, the wireless measurement system further includes an external data processing device configured to receive the first measurement data and the second measurement data.

In one or more embodiments of the present disclosure, the wireless measurement device is disposed on a fitted underwear.

In one or more embodiments of the present disclosure, the wireless measurement device transmits data by way of Bluetooth Low Energy.

In one or more embodiments of the present disclosure, the wireless measurement device is configured to measure an impedance signal.

In one or more embodiments of the present disclosure, the plurality of the measurement devices are multiple wireless measurement devices.

In one or more embodiments of the present disclosure, a front surface and a back surface of the wireless measurement device are configured with electrodes thereon respectively.

According to some embodiments of the present disclosure, a wireless measurement method for measuring data of human body by multiple measurement points is provided. The wireless measurement method includes: setting a wireless measurement device and a wearable measurement device with their corresponding parts of the human body and measurement modes; measuring and obtaining a first measurement data detected by the wireless measurement device and a second measurement data detected by the wearable measurement device; determining and classifying the first measurement data obtained by the wireless measurement device and the second measurement data obtained by the wearable measurement device; confirming which parts of the human body the first measurement data and the second measurement data obtained come from; transmitting the data of the human body obtained to the wearable measurement device or an external data processing device; and allowing a user to download the first measurement data and the second measurement data obtained.

It is to be understood that both the foregoing general description and the following detailed description are by examples, and are intended to provide further explanation of the disclosure as claimed.

### BRIEF DESCRIPTION OF THE DRAWINGS

The disclosure can be more fully understood by reading the following detailed description of the embodiment, with reference made to the accompanying drawings as follows:

FIG. 1 is a schematic diagram of a wireless measurement system for measuring data of human body by multiple measurement points according to some embodiments of the present disclosure;

FIG. 2 is a schematic diagram of an application of the wireless measurement device according to some embodiments of the present disclosure;

FIG. 3A is a schematic diagram of a front surface of the wireless measurement device according to some embodiments of the present disclosure;

FIG. 3B is a schematic diagram of a back surface of the wireless measurement device according to some embodiments of the present disclosure;

FIG. 4 is a schematic top view of the wearable measurement device according to some embodiments of the present disclosure;

FIG. 5 is a schematic flow chart of a wireless measurement method for measuring data of human body by multiple measurement points according to some embodiments of the present disclosure; and

FIG. 6 is a schematic diagram of a three-point measurement according to some embodiments of the present disclosure.

### DETAILED DESCRIPTION

Reference will now be made in detail to the present embodiments of the disclosure, examples of which are illustrated in the accompanying drawings. Wherever possible, the same reference numbers are used in the drawings and the description to refer to the same or like parts.

In various embodiments, the description is made with reference to figures. However, certain embodiments may be practiced without one or more of these specific details, or in combination with other known methods and configurations. In the following description, numerous specific details are set forth, such as specific configurations, dimensions, and processes, etc., in order to provide a thorough understanding of the present disclosure. Reference throughout this specification to “one embodiment,” “an embodiment” or the like means that a particular feature, structure, configuration, or characteristic described in connection with the embodiment is included in at least one embodiment of the disclosure. Thus, the appearances of the phrase “in one embodiment,” “in an embodiment” or the like in various places throughout this specification are not necessarily referring to the same embodiment of the disclosure. Furthermore, the particular features, structures, configurations, or characteristics may be combined in any suitable manner in one or more embodiments.

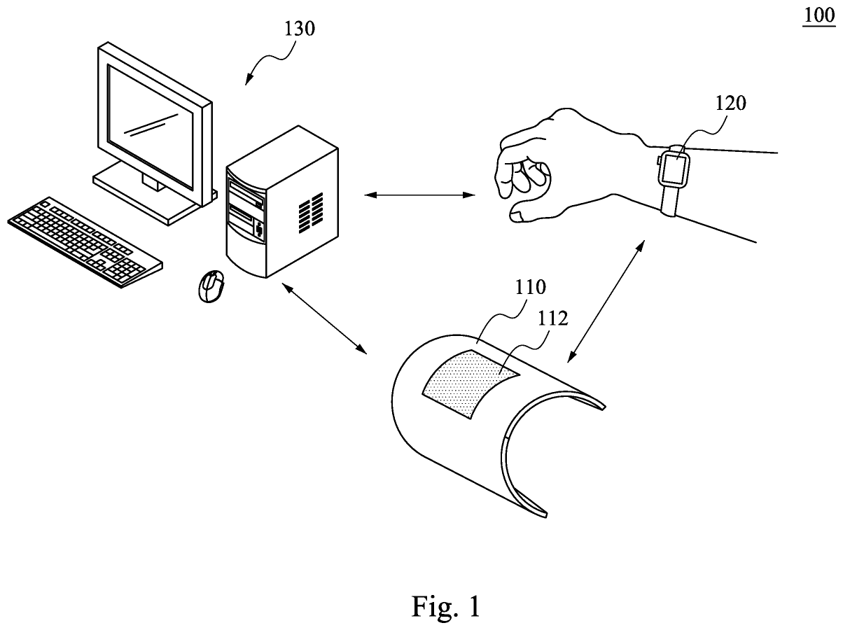

Reference is made to FIG. 1. FIG. 1 is a schematic diagram of a wireless measurement system 100 for measuring data of human body by multiple measurement points according to some embodiments of the present disclosure. The wireless measurement system 100 for measuring data of human body by multiple measurement points includes a plurality of measurement devices 110, 120. One of the plurality of the measurement devices 110, 120 is a wireless measurement device 110. The wireless measurement device 110 is configured to attach a skin of the human body and to obtain a first measurement data. The wireless measurement device 110 is further configured to perform an all-day measurement and to return the first measurement data with a fixed time period when the wireless measurement device 110 is attached to the skin of the human body. In some embodiments, the wireless measurement system 100 for measuring data of human body by multiple measurement points further includes an external data processing device 130 configured to receive the first measurement data. The external data processing device 130 may be a desktop computer as shown in FIG. 1, or it may be simplified as a mobile phone or another hardware which contains software capable of processing data.

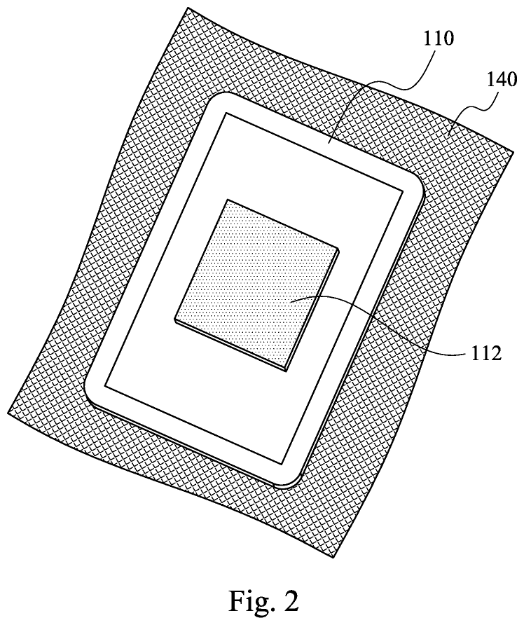

Reference is made to FIGS. 1 and 2. FIG. 2 is a schematic diagram of an application of the wireless measurement device 110 according to some embodiments of the present disclosure. In some embodiments, the wireless measurement device 110 is disposed on a fitted underwear 140. A user can wear the wireless measurement device 110 almost without burden. By an electrode 112 configured on the wireless measurement device 110 combined with a monitoring setting of 24-hour (all-day) monitoring, more complete and continuous data is obtained to monitor a condition of the human body. In some embodiments, the wireless measurement device 110 transmits data by way of Bluetooth Low Energy (BLE), such that wearing the wireless measurement device 110 for a long time without taking it off or charging it is allowable, and the convenience is greatly increased. In addition, since the wireless measurement device 110 can be set to return the measured data to the external data processing device 130 every other time period, the power can be further saved, and the working time can be maintained longer. The foregoing embodiments of the wireless measurement device 110 is of great convenience in measuring data of the human body such as heart rate variability (HRV) that needs to be tracked for a long time. The wireless measurement device 110 disposed on the fitted underwear 140 can be placed on a heart part to measure voltages, and may also be placed on different parts of the human body and configured with different types of sensors. For example, a sensor is placed on a thigh to measure impedance signals to obtain pressure signals of muscle for a long time. The pressure signal of muscle can be further used to determine whether there are symptoms such as muscle atrophy.

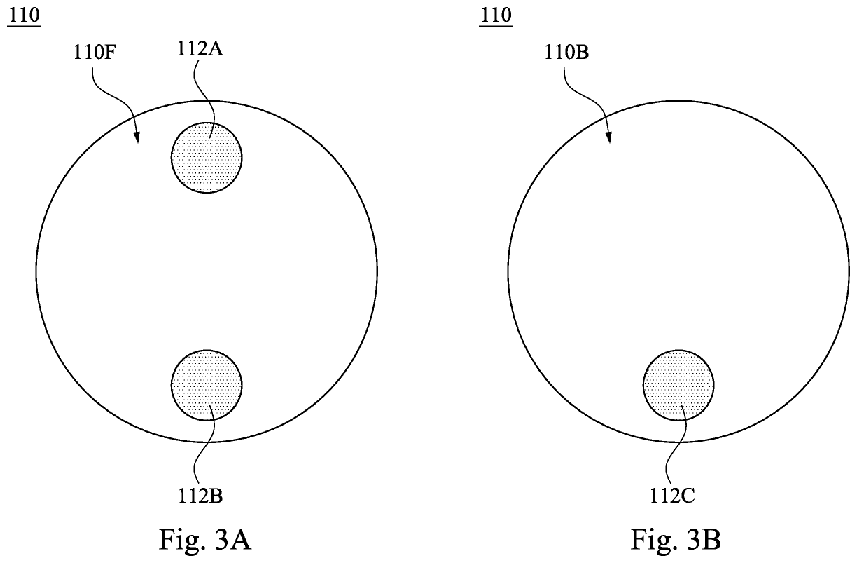

Reference is made to FIGS. 1, 3A, and 3B. FIG. 3A is a schematic diagram of a front surface 110F of the wireless measurement device 110 according to some embodiments of the present disclosure. FIG. 3B is a schematic diagram of a back surface 110B of the wireless measurement device 110 according to some embodiments of the present disclosure. The front surface 110F and the back surface 110B as mentioned are opposite surfaces of an object as is known for public. In some embodiments, the back surface 110B of the wireless measurement device 110 is configured to be directly attached to the skin of the human body. The electrode 112C contacts the skin of the human body. At the meantime, the front surface 110F of the wireless measurement device 110 faces away from the skin of the human body and faces outside the human body. The user can contact a part of the human body (except the part covered by the back surface 110B) to an electrode 112A and/or an electrode 112B provided on the front surface 110F of the wireless measurement device 110, so as to meet needs and to facilitate multiple points measurement. Since the user is allowed to choose any part of the human body to contact the electrode 112A and/or the electrode 112B, the selection of measurement sites can be very flexible and can be changed at any time without a need for complicated disassembly and installation steps.

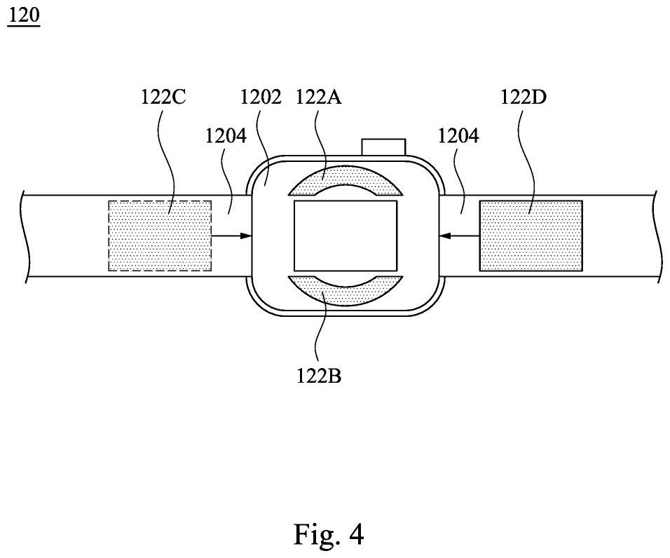

Reference is made to FIGS. 1 and 4. FIG. 4 is a schematic top view of the wearable measurement device 120 according to some embodiments of the present disclosure. In some embodiments, the plurality of the measurement devices 110, 120 include a wearable measurement device 120 configured to have data communication with the wireless measurement device 110. The wearable measurement device 120 may be a smart watch as shown in FIGS. 1 and 4, and may also be a ribbon, but should not be limited thereto. Any devices which is wearable and is configured to contact the skin of the human body does not depart from the scope of the wearable measurement device 120 disclosed in the present disclosure. In the embodiments illustrated by FIG. 4, the wearable measurement device 120 is exemplified as a (smart) watch. The wearable measurement device 120 includes a crown 1202 and a band 1204. The crown 1202 is provided with an electrode 122A and/or an electrode 122B thereon. The band 1204 is provided with an electrode 122 and/or an electrode 122D thereon. In some embodiments, the electrode 122A and the electrode 122B on the crown 1202 face away from the skin of the human body and faces outside the human body. The user can contact a part of the human body (except the part covered by the crown 1202) to the electrode 122A and/or the electrode 122B provided on the crown 1202 to meet needs and to facilitate multiple points measurement. At least one of the electrode 122C and the electrode 122D on the band 1204 is on a side of the band 1204 facing the human body (e.g., the electrode 122C as exemplified in FIG. 4). The electrode 122C is drawn with a dotted line to indicate that the electrode 122C is disposed on the other side with respect to a side as shown in FIG. 4. Therefore, when the user wear the wearable measurement device 120 as shown in FIG. 4, the electrode 122C contacts the human body and is able to perform measurements on the human body to obtain the second measurement data. The external data processing device 130 as mentioned may also be configured to receive the second measurement data.

In some embodiments, a wireless measurement device 110 is disposed on a chest of the user, and the first measurement data (i.e., an electrocardiography (ECG) data) is detected by the electrode 112. A wearable measurement device 120 is disposed on the wrist and the second measurement data (i.e., a pulse wave data) is detected by at least the electrode 122C. The first measurement data and the second measurement data are transmitted to the external data processing device 130. Alternatively, parameters of a blood pressure are obtained after the first measurement data and the second measurement data are analyzed by the wearable measurement device 120. The parameters of the blood pressure can be monitored for 24hours without disturbing the normal life of the user, so as to obtain a more complete parameters of the blood pressure which are closer to real physical conditions of the user. The above settings are useful for applications such as ECG, photoplethysmography (PPG) . . . etc., which can facilitate convenience of measurements and improve accuracy due to long-term monitoring. When the application is PPG, at least one of the wireless measurement device 110 and the wearable measurement device 120 includes a light-emitting diode and a photodiode. The light-emitting diode is configured to emit light to the skin of the human body. The photodiode is configured to receive light reflected by the skin of the human body. A figure of PPG is obtained by analyzing light intensity and variations of the light intensity. In addition, a pressure value with high accuracy can be obtained by combining ECG and PPG. In some embodiments, a body part sensor is configured on the wireless measuring device 110 to verify which part of the human body to which the human skin belongs.

In the embodiments of the present disclosure as mentioned, since the wireless measurement system 100 for measuring data of human body by multiple measurement points perform measurements by way of wireless communication, the wireless measurement device 110, the wearable measurement device 120, and the external data processing device 130 are able to communicate with one another wirelessly, and the user can move freely and may even leave an examination room in a hospital. In addition, with the convenience as mentioned and combined with a periodic transmission by the Bluetooth Low Energy, a length of a period for the periodic transmission can also be modified according to different measuring projects. For example, if a measurement on a heart rate, a premature beat, or a bradycardia/tachycardia is required, a length of a period for a periodic measurement or the periodic transmission may be set to be about 5 seconds. If a preventive measurement on an atrial fibrillation, a sudden cardiac death, a ventricular fibrillation, or an atrioventricular block is required, the length of the period for the periodic measurement or the periodic transmission may be set to be about 5 seconds. The length of the periods as disclosed above are merely an example, and the scope of the present disclosure is not limited thereto. If there is a need to reduce power consumption, the transmission can be performed after multiple sets of data are obtained. That is, a cycle length of the periodic transmission is an integer multiple of a cycle length of the periodic measurement.

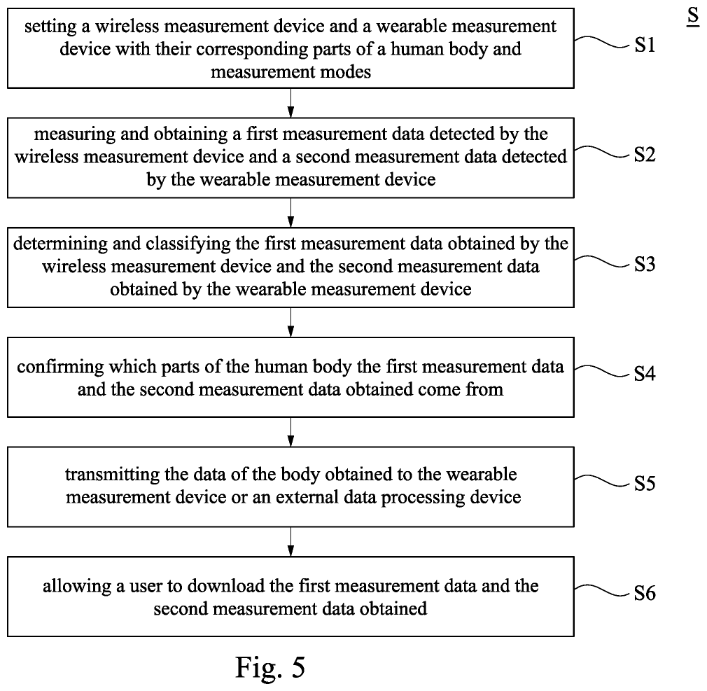

Reference is made simultaneously to FIGS. 1 and 5. FIG. 5 is a schematic flow chart of a wireless measurement method S for measuring data of human body by multiple measurement points according to some embodiments of the present disclosure. The wireless measurement method S includes the following steps of: setting the wireless measurement device 110 and the wearable measurement device 120 with their corresponding parts of a body and measurement modes (step S1); measuring and obtaining the first measurement data detected by the wireless measurement device 110 and the second measurement data detected by the wearable measurement device 120 (step S2); determining and classifying the first measurement data obtained by the wireless measurement device 110 and the second measurement data 120 obtained by the wearable measurement device (step S3); confirming which parts of the human body the first measurement data and the second measurement data obtained come from (step S4); transmitting the data of the human body obtained to the wearable measurement device 120 or an external data processing device 130 (step S5); and allowing a user to download the first measurement data and the second measurement data obtained (step S6).

The wireless measurement method S for measuring data of human body by multiple measurement points can be performed by the wireless measurement system 100 as mentioned. In step S1, setting the wireless measurement device 110 and the wearable measurement device 120 with their corresponding parts of a body and measurement modes may be to set the electrode 112 of the wireless measurement device 110 to correspond to the lower limb (e.g., a left leg), and to set the electrode 122C of the wearable measurement device 120 to correspond to the right hand. In some embodiments, a third measurement data corresponding to the left hand may be measured by another wireless measurement device added to a left hand, or by pressing the electrode 122A of the wearable measurement device 120 by the user. The measurement mode can be a mode which measures resistance, impedance, or light intensity described previously. In addition, the measurement mode also includes the aforementioned periodic measurement or settings of transmission.

Determining and classifying the first measurement data and the second measurement data in step S3 include arranging corresponding data structures for the first measurement data and the second measurement data according to the parts of the human body and the measurement modes as set. For example, if the first measurement data is measured once every 5 seconds and the second data is measured once every 1 second, the two sets of data must be labeled separately to facilitate cross references in a subsequent data processing. Step S3 can be accomplished by the external data processing device 130 or the wearable measurement device 120.

In step S4, in addition to directly referring to the settings in step S1 and confirming the parts of the human body corresponding to the first measurement data and the second measurement data, it is also possible to further confirm actual parts of the human body by the body part sensor pre-configured to verify which part of the human body to which the human skin belongs, so as to avoid misinterpretation of the parts of the human body caused by misuse of the user.

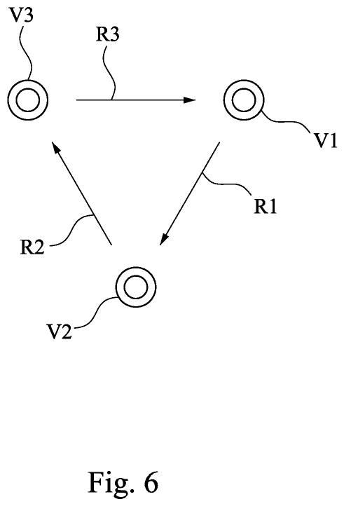

A specific way of the measurement for the embodiment mentioned above is, for example, a three-point measurement, but should not be limited thereto. Reference is made to FIG. 6. FIG. 6 is a schematic diagram of a three-point measurement according to some embodiments of the present disclosure. For example, three electrodes in the above embodiments (numbers of labeling are omitted herein) are respectively in contact with three limbs (e.g., a left hand, a right hand, and the left leg, but should not be limited thereto) among the four limbs, and to obtain the data of the human body. To simplify the description, all of the three measurement points measure voltages, which are a first voltage V1, a second voltage V2, and a third voltage V3, respectively. The first measurement data and the second measurement data as mentioned may correspond to two of the three voltages in the embodiments of FIG. 6. It is noted that the correspondences are an exemplification, and the first measurement data and the second measurement data are not limited thereto. In such embodiments, a difference between the second voltage V2 and the first voltage V1 can be measured and obtained through a first path R1, i.e., V2-V1; a difference between the third voltage V3 and the second voltage V2 can be measured and obtained through a second path R2, i.e., V3-V2; and a difference between the first voltage V1 and the third voltage V3 can be measured and obtained through a third path R3, i.e., V1-V3. Through the first path R1, the second path R2, and the third path R3, it can be measured whether a heart is functioning normally. For example, waveforms measured out include a P wave, a Q wave, an R wave, a S wave, and a T wave. These waveforms may be interpreted by professional doctors or trained persons, so as to obtain data related to the heart.

In summary, embodiments of the present disclosure disclose a wireless measurement system and a method thereof for measuring data of human body by multiple measurement points. Since the wireless measurement device, the wearable measurement device, and the external data processing device are able to communicate with one another wirelessly, the user can move freely and may even leave a specific examination room. In combination with the low-power periodic transmission, the user can easily monitor data of his/her own body for a long time.

Although the present disclosure has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the method and the structure of the present disclosure without departing from the scope or spirit of the disclosure. In view of the foregoing, it is intended that the present disclosure cover modifications and variations of this disclosure provided they fall within the scope of the following claims.

What is claimed is:

1. A wireless measurement system for measuring data of human body by multiple measurement points, comprising:
a plurality of measurement devices, one of the plurality of the measurement devices being a wireless measurement device configured to attach a skin of the human body and to obtain a first measurement data, wherein the wireless measurement device is further configured to perform an all-day measurement and to return the first measurement data with a fixed time period when attached to the skin of the human body.

2. The wireless measurement system of claim 1, wherein the plurality of the measurement devices comprise a wearable measurement device configured to have data communication with the wireless measurement device.

3. The wireless measurement system of claim 2, wherein the wearable measurement device is a ribbon or a smart watch that comprises at least one electrode thereon, and the at least one electrode is configured to contact the skin of the human body and to obtain a second measurement data.

4. The wireless measurement system of claim 3, wherein the wireless measurement system further comprises an external data processing device configured to receive the first measurement data and the second measurement data.

5. The wireless measurement system of claim 1, wherein the wireless measurement device is disposed on a fitted underwear.

6. The wireless measurement system of claim 1, wherein the wireless measurement device transmits data by way of Bluetooth Low Energy.

7. The wireless measurement system of claim 1, wherein the wireless measurement device is configured to measure an impedance signal.

8. The wireless measurement system of claim 1, wherein the plurality of the measurement devices are multiple wireless measurement devices.

9. The wireless measurement system of claim 1, wherein a front surface and a back surface of the wireless measurement device are configured with electrodes thereon respectively.

10. A wireless measurement method for measuring data of human body by multiple measurement points, comprising:
setting a wireless measurement device and a wearable measurement device with their corresponding parts of the human body and measurement modes;
measuring and obtaining a first measurement data detected by the wireless measurement device and a second measurement data detected by the wearable measurement device;
determining and classifying the first measurement data obtained by the wireless measurement device and the second measurement data obtained by the wearable measurement device;
confirming which parts of the human body the first measurement data and the second measurement data obtained come from;
transmitting the data of the human body obtained to the wearable measurement device or an external data processing device; and
allowing a user to download the first measurement data and the second measurement data obtained.
